(19) United States
(12) Patent Application Publication
(10) Pub. No.: US 2015/0066118 A1
(43) Pub. Date: Mar. 5, 2015

(54) Intravascular Neuromodulation Device Having a Spiral Track and Associated Methods

(71) Applicant: Medtronic Ardian Luxembourg S.a.r.l., Luxembourg (LU)

(72) Inventor: Barry O'Connell, Galway (IE)

(73) Assignee: Medtronic Ardian Luxembourg S.a.r.l., Luxembourg (LU)

(21) Appl. No.: 14/041,270

(22) Filed: Sep. 30, 2013

Related U.S. Application Data
(63) Continuation-in-part of application No. 14/012,431, filed on Aug. 28, 2013, now abandoned.

(51) Int. Cl. A61N 1/28

(52) U.S. Cl. CPC A61N 1/28
USPC 607/101

(57) ABSTRACT

Assemblies, systems, and methods for intravascular neuromodulation include a method of positioning a treatment device and a track element at a treatment site in an artery of a human patient and transforming the track element from a delivery configuration to a deployed configuration to form a spiral-shaped track tending to be in apposition with an inner wall of the artery. The treatment device has a distally-located neuromodulation element for sliding along the spiral-shaped track. The method can also include delivering energy via the neuromodulation element across the inner wall of a renal artery to heat or otherwise electrically modulate neural fibers that contribute to renal function.

### TECHNICAL FIELD

The present technology relates generally to intravascular neuromodulation and associated methods. In particular, several embodiments are directed to devices positionable along spiral tracks for intravascular renal neuromodulation and associated methods.

### BACKGROUND

The sympathetic nervous system (SNS) is a primarily involuntary bodily control system typically associated with stress responses. Fibers of the SNS innervate tissue in almost every organ system of the human body and can affect characteristics such as pupil diameter, gut motility, and urinary output. Such regulation can have adaptive utility in maintaining homeostasis or preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (“NE”) spillover rates in patients with essential hypertension.

Cardio-renal sympathetic nerve hyperactivity can be particularly pronounced in patients with heart failure. For example, an exaggerated NE overflow from the heart and kidneys of plasma is often found in these patients. Heightened SNS activation commonly characterizes both chronic and end stage renal disease. In patients with end stage renal disease, NE plasma levels above the median have been demonstrated to be predictive of cardiovascular diseases and several causes of death. This is also true for patients suffering from diabetic or contrast nephropathy. Evidence suggests that sensory afferent signals originating from diseased kidneys are major contributors to initiating and sustaining elevated central sympathetic outflow.

Sympathetic nerves innervating the kidneys terminate in the blood vessels, the juxtaglomerular apparatus, and the renal tubules. Stimulation of the renal sympathetic nerves can cause increased renin release, increased sodium (Na+) reabsorption, and a reduction of renal blood flow. These neural regulation components of renal function are considerably stimulated in disease states characterized by heightened sympathetic tone and likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others. Recently, intravascular devices that reduce sympathetic nerve activity by applying an energy field to a target site in the renal blood vessel (e.g., via radio frequency ablation) have been shown to reduce blood pressure in patients with treatment-resistant hypertension.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent. For ease of reference, throughout this disclosure identical reference numbers may be used to identify identical or at least generally similar or analogous components or features.

FIG. 1 is a partially schematic diagram of a neuromodulation system configured in accordance with an embodiment of the present technology.

FIG. 2 is a longitudinal cross-sectional view of an intravascular therapeutic assembly in a delivery state (e.g., low-profile or collapsed configuration) and carried within a delivery element in accordance with an embodiment of the present technology.

FIG. 3A is a perspective view of the intravascular therapeutic assembly of FIG. 2 having a spiral-shaped track in a deployed state (e.g., expanded configuration) within a renal artery of a patient in accordance with a further embodiment of the present technology.

FIG. 3B is a transverse cross-sectional view of the intravascular therapeutic assembly taken along line 3B-3B of FIG. 3A.

FIG. 4A is a transverse cross-sectional view of an intravascular therapeutic assembly configured in accordance with an embodiment of the present technology.

FIG. 4B is a transverse cross-sectional view of an intravascular therapeutic assembly configured in accordance with another embodiment of the present technology.

FIG. 5 schematically illustrates modulating renal nerves with an intravascular therapeutic assembly configured in accordance with an embodiment of the present technology.

FIG. 6A is a longitudinal cross-sectional view of an intravascular therapeutic assembly and guidewire in a delivery state (e.g., low-profile or collapsed configuration) in accordance with another embodiment of the present technology.

FIG. 6B is a perspective view of the intravascular therapeutic assembly of FIG. 6A with the guidewire partially withdrawn and showing the therapeutic assembly in a deployed state (e.g., expanded configuration) within a renal artery of a patient in accordance with a further embodiment of the present technology.

FIG. 7 is a flowchart of a method for delivering and deploying an intravascular therapeutic assembly in accordance with an embodiment of the present technology.

FIG. 8 is a flowchart of another method for delivering and deploying an intravascular therapeutic assembly in accordance with an embodiment of the present technology.

### DETAILED DESCRIPTION

The present technology is directed to apparatuses, and methods for achieving electrically- and/or thermally-induced renal neuromodulation (i.e., rendering neural fibers that innervate the kidney inert or inactive or otherwise completely or partially reduced in function) by percutaneous transluminal intravascular access. In particular, embodiments of the present technology relate to therapeutic assemblies having track elements and treatment devices (e.g., treatment catheters) slidably engaged with the track elements. The therapeutic assemblies include at least one neuromodulation element (e.g., at least one electrode) that can be located, for example, at a distal portion of the treatment device. After deployment in a target blood vessel of a human patient, a distal portion of the track element is transformable between a delivery or low-profile state (e.g., a generally straightened shape) to a deployed state (e.g., a radially expanded, generally spiral/helical shape) such that the track element defines a spiral-shaped track in apposition with an inner wall of the target blood vessel (e.g., renal artery).

The treatment device can include a treatment catheter or another elongate member that slidably engages the track element such that movement of the of the treatment device relative to the track element translates the neuromodulation element(s) along the track element to position the neuromodulation element(s) at various treatment positions within the target blood vessel. In one embodiment, for example, the track element can be a wire (e.g., nitinol wire) that is accommodated within a lumen of the treatment device (e.g., treatment catheter, microcatheter, tubular sheath, etc.) and has an expandable, pre-formed, helical shape at a distal portion thereof. Accordingly, movement of the treatment device proximally or distally along the deployed and stationary track element can displace the neuromodulation element both angularly or circumferentially and longitudinally relative to a longitudinal axis of the target blood vessel.

The neuromodulation element(s) are in electrical communication with an energy source or energy generator external to the patient such that energy is delivered via the neuromodulation element(s) to portions of a renal artery after being advanced thereto along a percutaneous transluminal path (e.g., a femoral artery puncture, an iliac artery and the aorta, a radial artery, or another suitable intravascular path). Suitable energy modalities include, for example, electrical energy, radio frequency (RF) energy, pulsed electrical energy, or thermal energy. The treatment device carrying the neuromodulation element(s) can be sized, shaped and have suitable flexibility such that the neuromodulation element(s) are in constant apposition with the interior wall of the renal artery when the track element is in the deployed (e.g., spiral/helical) state. The pre-formed spiral/helical shape of the deployed portion of the track element carrying the treatment device allows blood to flow through the assembly during therapy, which is expected to help prevent occlusion of the renal artery during activation of the neuromodulation element(s).

Previous energy-delivery catheter systems for inducing neuromodulation that include arrays of electrodes can be expensive to manufacture. For example, multiple electrodes require separate wiring of each electrode as well as complex algorithms and design of the energy generator. Additionally, repositioning and specific lesion placement on the interior wall of the renal artery are challenging and time consuming when using conventional energy-delivery catheter systems. In contrast, a self-expanding spiral frame over which an elongate member (e.g., sheath, treatment catheter, microcatheter, etc.) can travel provides a simple design that is easy to deploy and use compared to the conventional catheter devices. Moreover, the neuromodulation element can, in some embodiments, include a single electrode that can be moved proximally or distally along the track element while the track element remains in situ. The movement can be achieved via a pull or push mechanism that slides the neuromodulation element along the spiral-shaped track to easily select and access new ablation or treatment locations. Additionally, because movement of the neuromodulation element along the track element is achievable to access a plurality of treatment locations (both circumferentially and longitudinally displaced from each other), a single neuromodulation element can be deployed on the treatment device. This design aspect avoids the separate wiring that multiple electrodes would require, which is expected to reduce manufacturing time and material costs associated with additional separate electrodes and wiring, as well as reduce the complexity of the control algorithm typically necessary to operate more than one independent electrode or energy delivery elements.

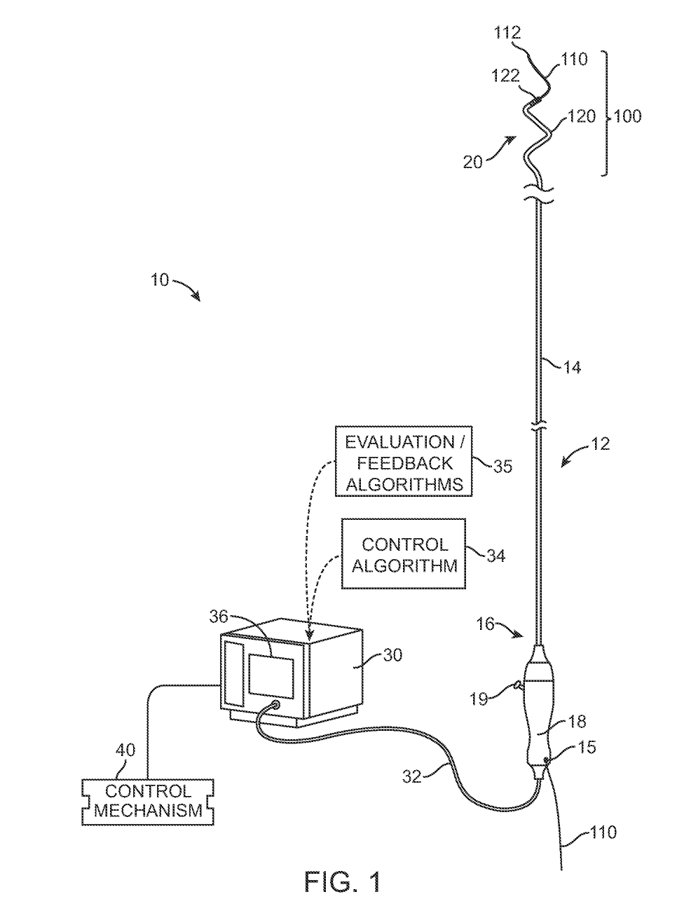

Specific details of several embodiments of the technology are described below with reference to FIGS. 1-7. Although many of the embodiments are described below with respect to devices, systems, and methods for intravascular modulation of renal nerves using spiral-shaped track elements, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-7.

As used herein, the terms “distal” and “proximal” define a position or direction with respect to the treating clinician or clinician's control device (e.g., a handle assembly). “Distal” or “distally” are a position distant from or in a direction away from the clinician or clinician's control device. “Proximal” and “proximally” are a position near or in a direction toward the clinician or clinician's control device.

### Selected Examples of Neuromodulation Systems

FIG. 1 is a partially schematic illustration of a renal neuromodulation system 10 (“system 10”) configured in accordance with an embodiment of the present technology. The system 10 includes an intravascular catheter 12 operably coupled to an energy source or energy generator 30 (e.g., a RF energy generator). The catheter 12 can include an elongated shaft 14 having a proximal portion 16 and a distal portion 20. The catheter 12 also includes a handle 18 at the proximal portion 16. The catheter 12 can further include a therapeutic assembly or treatment section 100 (shown schematically) at the distal portion 20 (e.g., attached to the distal portion 20, defining a section of the distal portion 20, etc.). As explained in further detail below, the therapeutic assembly 100 can include a treatment device 120 slidably engaging a distal portion of track element 110. In the exemplary over-the-wire (“OTW”) embodiment shown in FIG. 1, the proximal end of track element 110 extends from an exit port 15 in handle 18. The treatment device 120 can be an elongate member (e.g., a sheath, a treatment catheter, etc.) with one or more neuromodulation elements or energy delivery elements 122 disposed at a distal end thereof. In one embodiment, the neuromodulation or energy delivery element 122 can be an electrode for delivering energy at a target treatment site and providing therapeutically-effective electrically- and/or thermally-induced renal neuromodulation.

As explained in greater detail below, the therapeutic assembly 100 is configured to be intravascularly delivered to a target blood vessel (e.g., a renal blood vessel) of a human patient in a low-profile configuration. Upon delivery to the target treatment site, the therapeutic assembly 100 is further configured to be transformed into an expanded state (e.g., the distal portion of track element 110 is deployed into a generally spiral/helical configuration as shown schematically in FIG. 1) to place the track element 110 into apposition with an inner wall of the blood vessel.

Alternatively, the deployed state may be non-spiral provided that the deployed state places the track element and specifically one or more energy delivery elements 122 in vessel wall apposition for delivering the energy to the treatment site. The treatment device 120 can then be slidably moved along the track element 110 to position the neuromodulation or energy delivery element 122 at desired location(s) for modulating target nerves proximate to the inner wall of the blood vessel, thereby providing therapeutically-effective electrically- and/or thermally-induced renal neuromodulation.

The therapeutic assembly 100 may be transformed between the delivery and deployed states using a variety of suitable mechanisms or techniques (e.g., self-expansion). In one specific example, the distal portion of track element 110 can be a pre-formed, self-expanding wire that will transform into the deployed state or arrangement when unrestricted (e.g., by retracting a guide catheter, straightening sheath, etc.).

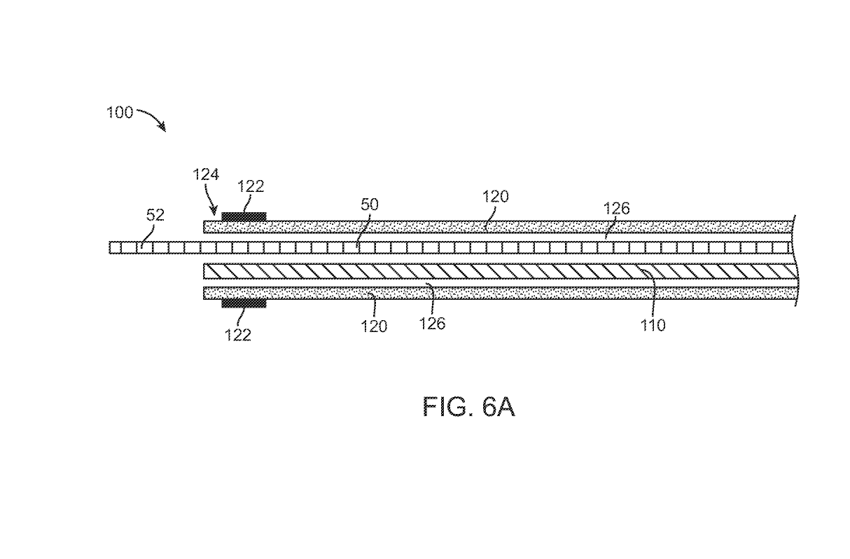

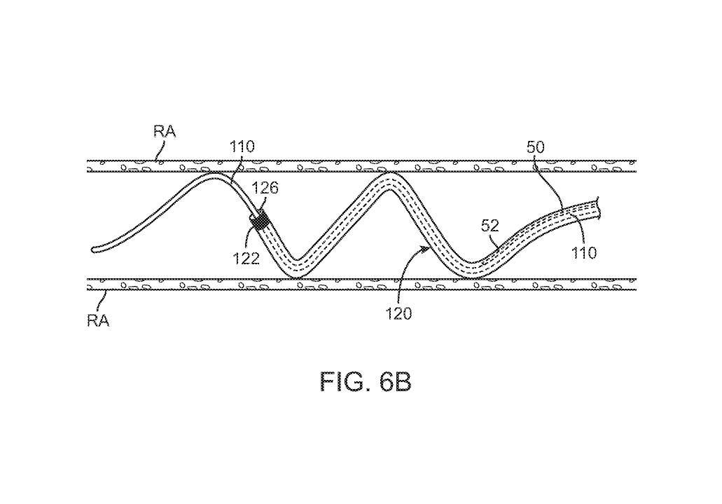

The proximal end of the treatment device 120 is carried by or affixed to the distal portion 20 of the elongated shaft 14. A distal end of the track element 110 may include an atraumatic tip 112. In some embodiments, the distal end of the catheter 12 may include an atraumatic tip for preventing intravascular trauma during delivery of the therapeutic assembly 100 to the treatment site. The distal end of the catheter 12 may also be configured to engage another element of the system 10 or catheter 12. For example, the distal end of the catheter 12 may define a passageway for receiving a guidewire for delivery of the treatment device using OTW or rapid exchange (“RX”) techniques. Further details regarding such arrangements are described below with reference to FIGS. 6A and 6B.

The neuromodulation element(s) 122 can be electrically coupled to the energy source 30 via a cable 32, and the energy source 30 (e.g., a RF energy generator) can be configured to produce a selected modality and magnitude of energy for delivery to the treatment site via the neuromodulation element 122 carried by the treatment device 120. As described in greater detail below, one or more supply wires (not shown) can extend along the elongated shaft 14 or through a lumen in the shaft 14 to the therapeutic assembly 100 and supply the treatment energy to the neuromodulation element 122.

A control mechanism 40, such as foot pedal or handheld remote control device, may be connected to the energy source 30 to allow the clinician to initiate, terminate and, optionally, adjust various operational characteristics of the energy source 30, including, but not limited to, power delivery. The remote control device can be positioned in a sterile field and operably coupled to the therapeutic assembly 100, and specifically to the neuromodulation element 122, and can be configured to allow the clinician to activate and deactivate the energy delivery to the neuromodulation element 122. In other embodiments, the remote control device may be built into the handle assembly 18.

The energy source or energy generator 30 can be configured to deliver the treatment energy via an automated control algorithm 34 and/or under the control of a clinician. For example, the energy source 30 can include computing devices (e.g., personal computers, server computers, tablets, etc.) having processing circuitry (e.g., a microprocessor) that is configured to execute stored instructions relating to the control algorithm 34. In addition, the processing circuitry may be configured to execute one or more evaluation/feedback algorithms 35, which can be communicated to the clinician. For example, the energy source 30 can include a monitor or display 36 and/or associated features that are configured to provide visual, audio, or other indications of power levels, sensor data, and/or other feedback. The energy source 30 can also be configured to communicate the feedback and other information to another device, such as a monitor in a catheterization laboratory.

The system 10 can also include one or more additional sensors (not shown) located proximate to or within the neuromodulation element 122. For example, the system 10 can include temperature sensors (e.g., additional thermocouples, thermistors, etc.), impedance sensors, pressure sensors, optical sensors, flow sensors, and/or other suitable sensors connected to one or more supply wires (not shown) that transmit signals from the sensors and/or convey energy to the therapeutic assembly 100.

### Selected Examples of Therapeutic Assemblies and Related Devices

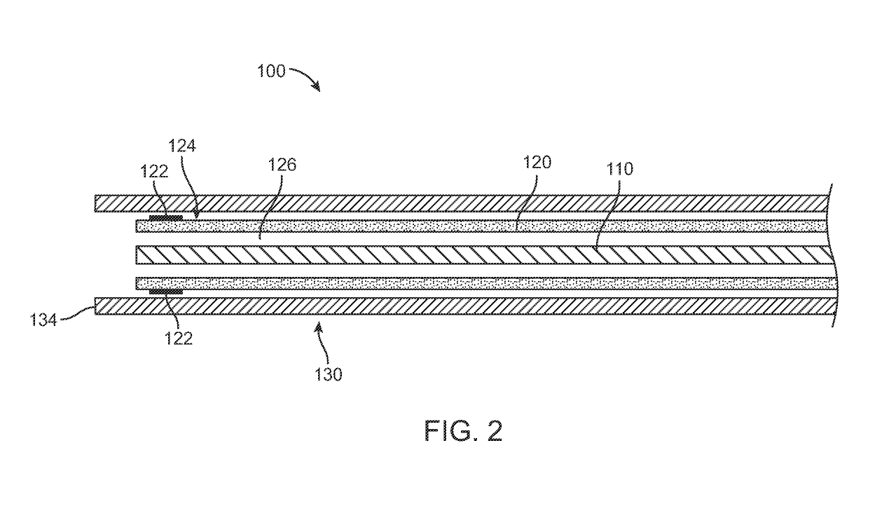

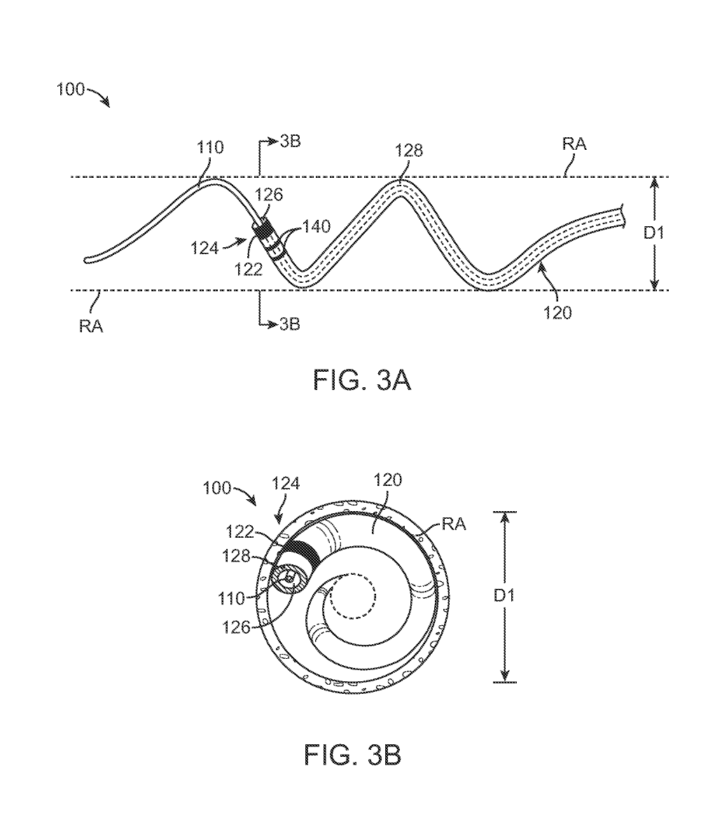

FIG. 2 is an longitudinal cross-sectional view of a portion of the intravascular therapeutic assembly 100 in a delivery state (e.g., a low-profile or collapsed configuration) in accordance with an embodiment of the present technology, and FIG. 3A is a perspective view of the therapeutic assembly 100 of FIG. 2 in a deployed state or expanded configuration within a renal artery RA (or other target blood vessel) of a patient. As noted above, the therapeutic assembly 100 can be transformed or actuated between the delivery state (FIG. 2) and the deployed state (e.g., a radially expanded, generally spiral/helical configuration, FIG. 3A).

Referring to FIGS. 2 and 3A together, the therapeutic assembly 100 includes the treatment device 120 carried by and slidably engaged with the distal portion of track element 110. The treatment device 120 includes the neuromodulation element 122 positioned at or near a distal portion 124 thereof for delivering therapeutically effective energy to target tissue (e.g., one or more nerves) of the patient. In the deployed state, the therapeutic assembly 100 is configured to place the distal portion of track element 110 and neuromodulation element 122 in apposition with an interior wall of the renal artery RA. The treatment device 120 comprises a treatment catheter, a microcatheter, or other elongate member (e.g., a sheath) having a lumen 126 therethrough defining a passageway for receiving the track element 110. The track element 110 accordingly provides a guide for the treatment device 120 as it moves over the track element 110.

Referring to FIG. 2, and in one embodiment, the therapeutic assembly 100 may be restrained in the delivery state (e.g., a generally straight or collapsed configuration) within the lumen of a tubular sheath or delivery element 130. The delivery element 130 can have a suitable radial and bending stiffness for restraining the distal portion of track element 110 in the low-profile and generally straight or non-spiral configuration (e.g., the delivery state). In some embodiments, for example, the delivery element 130 may comprise a guide catheter or a straightening sheath sized and shaped to restrain one or more components of the therapeutic assembly 100 in the low-profile state for delivery to the target treatment site within the renal artery RA. In the collapsed, low-profile configuration, the geometry of the therapeutic assembly 100 is configured to facilitate movement through the delivery element 130 to the treatment site. Persons of skill in the art of catheters will understand that delivery element 130, if it is a guide catheter, will typically have a pre-formed curved region (not shown) near the distal end. Thus, the delivery state of the therapeutic assembly 100 will be reduced, i.e. “low” in transverse profile, but not necessarily straight as it passes through the curved region of the guide catheter. Therapeutic assembly 100, in the low-profile configuration is sufficiently flexible to pass through the guide catheter, including the curved region. In some embodiments, the delivery element 130 may be 8 Fr or smaller (e.g., a 6 Fr guide catheter) to accommodate small renal arteries during delivery of the therapeutic assembly 100 to the treatment site. In other embodiments, however, the delivery element 130 may have a different size.

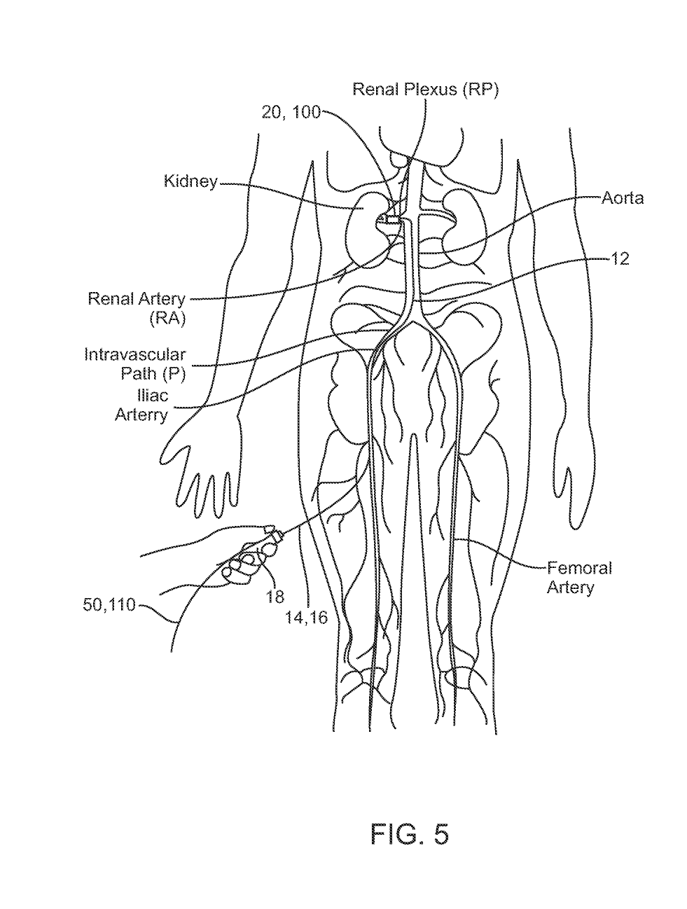

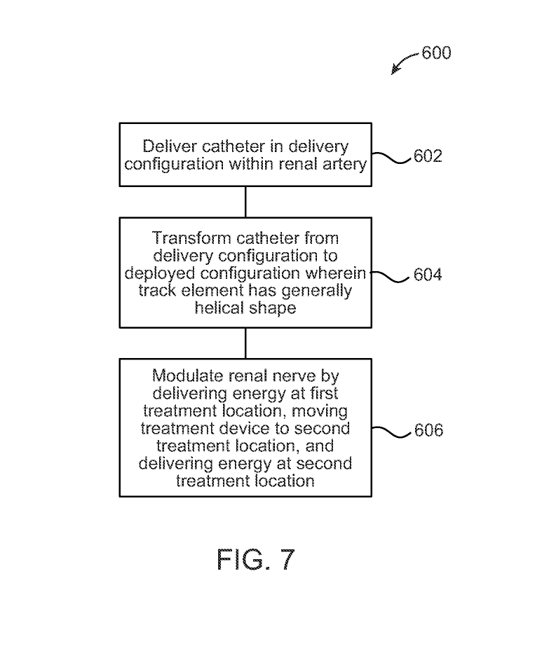

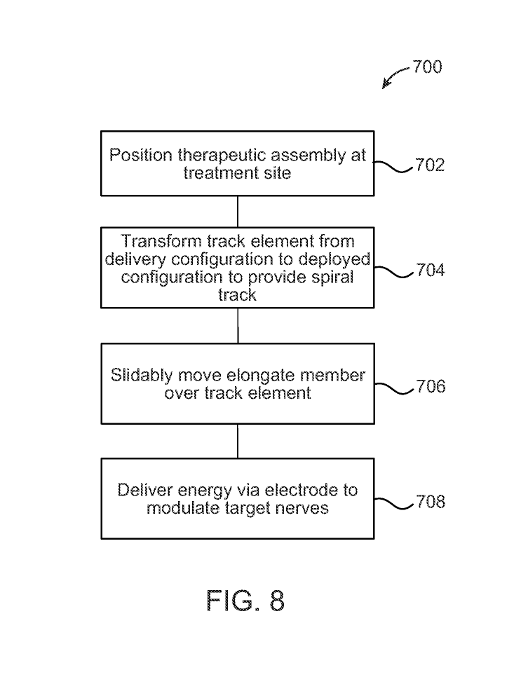

In some methods of using the neuromodulation system 10, the intravascular catheter 12 may be delivered and deployed over a guidewire 50 (shown in FIG. 5), rather than with a delivery element 130. The guidewire 50 can then be exchanged within lumen 126 for the track element 110, the pre-formed distal portion of which will tend to take its helical shape in the treatment site, thereby deforming the treatment device 120 into a similar helical shape such that at least the neuromodulation element 122 is placed in apposition with an interior wall of the renal artery RA. After forming suitable treatment zones or lesions on the inner wall of renal artery RA, the track element 110 may be partially or fully withdrawn or exchanged within lumen 126 for guidewire 50 to allow treatment device 120 to relax to a straighter configuration for removal from the patient. One of ordinary skill in the art will recognize a plurality of delivery protocols for delivering and deploying the therapeutic assembly 100 at the target treatment site, several of which are described further below and with respect to FIGS. 7 and 8.

As best seen in FIG. 3A, after delivery to the target treatment site (e.g. renal artery RA), the distal portion of track element 110 of the therapeutic assembly 100 may be deployed to its expanded, spiral-shaped configuration. In one embodiment, for example, the distal portion of track element 110 may be deployed by moving the delivery element 130 (e.g., sheath; FIG. 2) and therapeutic assembly 100 relative to each other such that the therapeutic assembly 100 including the distal portion of track element 110 is exposed distally beyond the delivery element 130. The distal portion of track element 110 can be configured to assume the expanded configuration when in an unbiased (e.g., unrestrained) condition. The delivery element 130 (FIG. 2), for example, can be pulled proximally while the therapeutic assembly 100 is held stationary with respect to the treatment site. Alternatively, the therapeutic assembly 100 can be pushed distally beyond a distal end 134 of the delivery element 130 while the delivery element 130 is held stationary with respect to the treatment site.

The spiral-shaped configuration of the distal portion of track element 110 is further illustrated in FIG. 3B, which is a transverse cross-sectional view of the therapeutic assembly 100 along the line 3B-3B of FIG. 3A. Referring to FIGS. 3A and 3B together, the distal portion of track element 110 defines a spiral track tending to contact the interior wall of the renal artery RA. In one embodiment, the distal portion of track element 110 can have a pre-set spiral/helical configuration such that the distal portion of track element 110 self-expands to a deployed geometry within the renal artery RA. The helix of track element 110 has a transverse dimension about its central axis CA that is at least approximately equal to a renal artery inner diameter D₁ (FIGS. 3A and 3B) and a maximum length in the direction of the central axis CA that is preferably less than or can be accommodated by the renal artery length (not shown). In other embodiments, however, the track element 110 may have a different arrangement and/or different dimensions.

In one embodiment, the track element 110 may be formed from suitable shape memory material, such as nitinol (nickel-titanium alloy) wire. In other embodiments, however, the track element 110 can be composed of different materials and/or have a different arrangement. For example, the track element 110 may be formed from other suitable materials such as metal wire (e.g., stainless steel), shape memory polymers, electro-active polymers, etc., that are pre-formed or pre-shaped into the desired deployed state (FIGS. 3A and 3B). Alternatively, the track element 110 may be formed from multiple materials such as a composite of one or more polymers and metals.

Referring again to FIGS. 3A and 3B together, the treatment device 120 is disposed over the distal portion of track element 110 such that, when the distal portion of track element 110 assumes its pre-set spiral/helical shape, the treatment device 120 is sufficiently flexible to conform to the helical shape of track element 110 such that at least neuromodulation element 122 can be placed in apposition with the interior wall of the renal artery RA. Treatment device 120 is moveable over the relatively stationary track element 110 for positioning or re-positioning element 122 at one or more treatment locations along the interior wall of the renal artery RA. In one embodiment, for example, the treatment device 120 can include a flexible tube 128 and at least the pre-shaped spiral/helical distal portion of track element 110 can be received within the lumen 126 of tube 128. The flexible tube 128 may be composed of a polymer material such as polyamide, polyimide, polyether block amide copolymer sold under the trademark PEBAX, polyethylene terephthalate (PET), polypropylene, an aliphatic, polycarbonate-based thermoplastic polyurethane sold under the trademark CARBOTHANE, or a polyether ether ketone (PEEK) polymer. The material properties and dimensions of the tube 128 are selected to provide the necessary flexibility for the tube 128 to readily deform between a relaxed, substantially straight shape and a shape that conforms to the helical deployed shape of the distal portion of track element 110. In other words, the tube 128 is more flexible than the track element 110 such that the shape of the combined components is defined by the shape of the track element 110. In some embodiments, the lumen 126 is sized to provide sufficient clearance with the track element 110 to reduce friction between the catheter 12 and the track element 110. In various embodiments, a lubricant or lubricious coating (not shown) can be included on either or both of the sliding surfaces, or may be applied between the track element 110 and the catheter 12 to facilitate relative movement therebetween.

In an alternative embodiment, the catheter 12 may include an operative wire (not shown) to facilitate pushing or pulling the treatment device 120 relative to the track element 110. The operative wire can extend proximally from the treatment device 120 (e.g. as an alternative to the shaft 14) to be accessible, for example, to a clinician outside the patient when the therapeutic assembly 100 is being delivered and deployed. In other embodiments, however, the treatment device 120 may have a different arrangement and/or different features.

In one embodiment, the neuromodulation element 122 can be an electrode configured to deliver energy (e.g., electrical energy, RF energy, pulsed electrical energy, non-pulsed electrical energy, thermal energy, etc.) across the wall of the renal artery RA. In a specific embodiment, the neuromodulation element 122 can deliver a thermal RF field to targeted renal nerves adjacent the wall of the renal artery RA. Referring to FIGS. 2-3B together, the neuromodulation element 122 can include a band electrode surrounding the distal end 124 of the treatment device 120 such as at the distal end of the flexible tube 128. For example, the neuromodulation element 122 can be a band electrode bonded to the tube 128 using an adhesive. In other embodiments, the treatment device 120 can include more than one neuromodulation element 122. For example, the device 120 may include 2 or 3 electrodes (not shown) or, in yet another embodiment, an array of electrodes such as a series of separate band electrodes spaced along the treatment device 120 and bonded to the flexible tube 128. Although band or tubular electrodes are illustrated, in other embodiments disc or flat electrodes may also be employed. In still another embodiment, electrodes having a spiral or coil shape may be utilized. The neuromodulation element 122 may be formed from any suitable metallic material (e.g., gold, platinum, an alloy of platinum and iridium, etc.). In other embodiments, however, the number, arrangement, and/or composition of the neuromodulation element(s) 122 may vary. For example, the neuromodulation element 122 may be placed on the treatment device 120 at another location proximal to the distal portion 124 of the treatment device 120.

The neuromodulation element 122 is electrically connected to an external energy source (such as energy source 30, FIG. 1) by a conductor or bifilar wire (not shown) extending through catheter 12. The neuromodulation element 122 may be welded or otherwise electrically coupled to its energy supply wire, and the wire can extend the entire length of the catheter 12 (e.g. inside, outside or within a wall of the treatment device 120 and shaft 14) such that a proximal end thereof is coupled to the energy source 30 (FIG. 1). In some embodiments, the therapeutic assembly 100 may also include an insulating layer (e.g., a layer of PET or another suitable material) over the track element 110 to further electrically isolate the material (e.g., nitinol wire) of the track element 110 from the wires (not shown).

In some embodiments, the therapeutic assembly 100 can include radiopaque markers 140 or other indicia for facilitating navigation of the assembly 100 through the vasculature as well as positioning of the neuromodulation element 122 at one or more desired treatment locations within the renal artery RA using x-ray imaging techniques known in the art. FIG. 3A illustrates an embodiment where radiopaque markers 140 are mounted to an outer surface of the distal portion 124 of the treatment device 120. In other embodiments, not shown, the track element 110 can also include radiopaque markers 140 (e.g., made with radiopaque ink). In certain aspects, at least a portion of the track element 110 and/or the treatment device 120 can be made from platinum and/or other radiopaque materials (e.g., platinum/iridium alloy metal coil wrapped around the elongate member).

In operation and referring to FIGS. 1-3B together, after the distal portion of track element 110 is self-expanded or otherwise deployed to its pre-set spiral/helical configuration in apposition with the interior wall of the renal artery RA, the treatment device 120 can be slid in either a proximal or distal direction along the relatively stationary track element 110 to position the neuromodulation element 122 at a desired treatment location. Therapeutically-effective energy can then be delivered via the neuromodulation element 122 across the wall of the renal artery RA to targeted renal nerves (not shown) at one or more treatment locations. For example, neuromodulatory energy can be delivered at a first treatment location followed by sliding the treatment device 120 in either a proximal or distal direction along the track element 110 to place the neuromodulation element 122 at a second treatment location transposed circumferentially and longitudinally offset from the first treatment location along the renal artery RA. This step can be followed by delivering energy via the neuromodulation element 122 across the wall of the renal artery RA to targeted renal nerves at the second treatment location. Further steps can include sliding the treatment device 120 along the track element 110 to one or more additional treatment locations and again delivering energy to target nerves.

In some embodiments, the second treatment location can be longitudinally spaced away from the first treatment location along the renal artery RA in either a proximal or distal direction. Sliding the treatment device 120 between first and second treatment locations can also translate the second treatment location circumferentially about the interior wall of the blood vessel with respect to the first treatment location. For example, as the treatment device 120 slides along the spiral-shaped track provided by the distal portion of the track element 110 (FIG. 3A), the neuromodulation element 122 is transposed circumferentially and longitudinally on the interior wall of the renal artery RA. Accordingly, energy can be delivered at one or more discrete treatment locations to form a helical pattern of lesions or treatment zones along the interior wall of the renal artery. In some embodiments, the treatment zones may overlap. In other embodiments, however, the lesions may be spaced sufficiently such that they do not overlap. In still further embodiments, energy can be delivered while sliding the treatment device 120 along the spiral-shaped track element 110 to form a continuous or approximately continuous helical lesion.

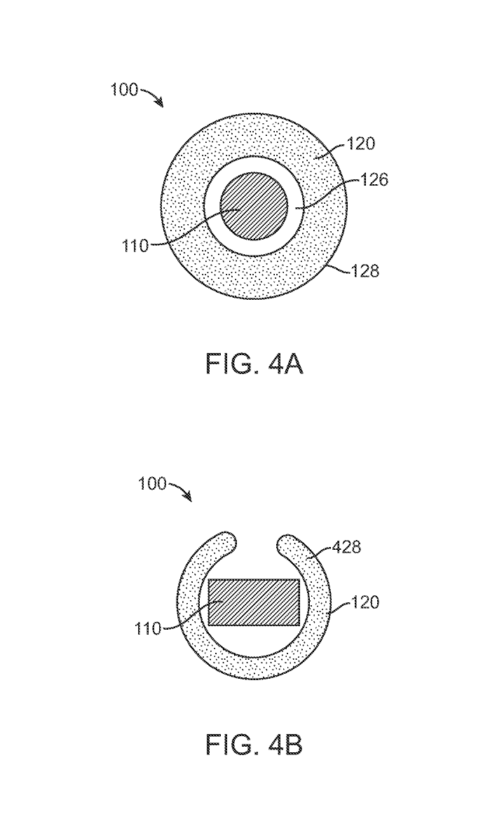

FIGS. 4A and 4B are transverse cross-sectional views of the therapeutic assembly 100 illustrating various arrangements of the treatment device 120 surrounding the track element 110. In one arrangement, and as shown in FIG. 4A, the track element 110 can be a wire having a generally circular cross-sectional shape, and the treatment device 120 can be an elongate member with a generally circular cross-sectional shape and having the lumen 126 for accommodating the track element 110. In this arrangement, the treatment device 120 may be a treatment catheter or other microcatheter having the flexible tube 128 that is configured to slidably engage the track element 110. FIG. 4B, however, shows an alternative arrangement wherein the track element 110 has a generally rectangular cross-sectional shape. FIG. 4B also depicts an embodiment of the treatment device 120 that does not include a tube 128, but instead incorporates a non-circumferential sleeve 428 that only partially encloses the track element 110. In addition to the arrangements shown in FIGS. 4A and 4B, those of ordinary skill in the art will recognize that there may be other suitable arrangements for providing a track or rail configured to be placed in apposition with the interior wall of the target blood vessel and a treatment device configured to engage and slidably move along the track or rail to position a neuromodulation element coupled thereto at a plurality of treatment locations.

### Selected Examples of Methods for Delivery and Deployment of Therapeutic Assemblies

Several suitable delivery methods are disclosed herein and discussed further below; however, one of ordinary skill in the art will recognize a plurality of methods suitable to deliver the therapeutic assembly 100 to the treatment site and to deploy the distal portion of track element 110 from the delivery configuration to the deployed configuration. With respect to the embodiment illustrated in FIGS. 1-3B, the track element 110 may be delivered to the treatment site through or within a guide catheter or straightening sheath (e.g., the delivery element 130 shown in FIG. 2). The sheath may be pre-placed across the treatment site with or without use of a guidewire, and the track element 110, with or without catheter 12 mounted thereabout, can then be passed through the sheath. Alternatively, track element 110 with or without catheter 12 mounted thereabout can be pre-loaded into the sheath such that the assembled components can be advanced simultaneously through the patient's vasculature. In any case, during delivery, the straightening sheath can partially or fully restrain the distal portion of track element 110 in the delivery configuration. When the distal portion of track element 110 is within the target site, the straightening sheath may be at least partially withdrawn or retracted to permit the distal portion of track element 110 to transform into the deployed configuration.

FIG. 5 (with additional reference to FIG. 1) illustrates at least one step of modulating renal nerves with an embodiment of the system 10. The therapeutic assembly 100 is shown positioned within the renal plexus RP and catheter 12 is shown in an intravascular path P extending from a percutaneous access site in a femoral (illustrated), brachial, radial, or axillary artery to a targeted treatment site within a respective renal artery RA. As illustrated, a section of the proximal portion 16 of the catheter shaft 14 is exposed externally of the patient even as the therapeutic assembly 100 has been advanced fully to the targeted treatment site in the patient. By manipulating the proximal portion 16 of the shaft 14 from outside the intravascular path P, the clinician may advance the shaft 14 through the sometimes tortuous intravascular path P and remotely manipulate the distal portion 20 of the shaft 14.

In the method step illustrated in FIG. 5, the therapeutic assembly 100 extends intravascularly to the treatment site over a guidewire 50 using an OTW technique. The guidewire 50 may comprise any suitable medical guidewire sized to slidably fit within the lumen 126 of catheter 12. In one particular embodiment, for example, the guidewire 50 may have a diameter of 0.356 mm (0.014 inch). When the guidewire 50 is used for delivery of the therapeutic assembly 100 to the treatment site, deployment of the track element 110 can be accomplished by exchanging the guidewire 50 for the track element 110 within the lumen 126 of the catheter 12. This exchange can be accomplished through the open proximal end of lumen 126 at exit port 15 in handle 18 (See FIG. 1). Thus, in FIG. 5, the exposed element is labeled as either guidewire 50 or track element 110.

In another method of delivery, FIG. 6A illustrates a transverse cross-sectional view of a portion of the intravascular therapeutic assembly 100 wherein both a guidewire 50 and track element 110 are disposed within treatment device 120. FIG. 6B is a perspective view of the therapeutic assembly 100 of FIG. 6A in a deployed state (e.g., expanded configuration) within a renal artery RA (or other target blood vessel) of a patient. As illustrated, both guidewire 50 and track element 110 are slidably disposed in lumen 126, which will necessarily be somewhat larger than the earlier embodiment wherein only one of guidewire 50 or track element 110 are present in lumen 126. In another embodiment (not shown) guidewire 50 and track element 110 can be slidably disposed in separate, dedicated lumens. The guidewire 50 may be sufficiently stiff to keep treatment device 120 relatively straight and thereby restrain the track element 110 in the delivery state. It will be understood that, without additional bending stiffness provided by either guidewire 50 or delivery element 130 (of the previous method), treatment device 120 will tend to conform to the shape of track element 110. When the guidewire 50 is partially refracted or withdrawn from the treatment site, as illustrated in FIG. 6B, the track element 110 provides a shape-recovery force sufficient to overcome the straightening force provided by a distalmost portion 52 of the guidewire 50 such that the track element 110 can deploy into its spiral/helical shaped configuration and deform treatment device 120 along with it. Further, because the distalmost portion 52 of the guidewire 50 can remain at least partially within the therapeutic assembly 100 while in the deployed state (e.g., FIG. 6B), the guidewire 50 can impart additional structural integrity to the positioning of the spiral-shaped portion during treatment. This feature is expected to help mitigate or reduce problems associated with keeping the therapeutic assembly 100 in place during treatment (e.g., help with vasoconstriction).

In an alternate method step, the guidewire 50 including the distalmost portion 52 may be withdrawn completely from the therapeutic assembly 100 while remaining within the shaft 14 (not shown) to permit the transformation of therapeutic assembly 100. In yet another method step, the guidewire 50 may be withdrawn completely from the shaft 14. In any of the foregoing examples, the clinician can withdraw the guidewire 50 sufficiently to observe transformation of the therapeutic assembly 100 to the deployed configuration and/or until an X-ray image shows that the distal tip of the guidewire 50 is at a desired location relative to the therapeutic assembly 100 (e.g., at least partially withdrawn from the therapeutic assembly 100, or completely withdrawn from the therapeutic assembly 100, etc.). In some methods, the extent of withdrawal of the guidewire 50 can be based, at least in part, on the clinician's judgment with respect to the selected guidewire and the extent of withdrawal necessary to achieve deployment of the therapeutic assembly 100.

After formation of lesions or treatment zones suitable for achieving neuromodulation, and in accordance with one method, the therapeutic assembly 100 may be transformed back to the low-profile delivery configuration by axially advancing the guidewire 50 relative to the therapeutic assembly 100 (e.g., within the lumen 126 of the treatment device 120). Following advancement of the guidewire 50, the track element 110 can be withdrawn from the renal artery RA, or in another embodiment, the guidewire 50 can be exchanged for the track element 110 in lumen 126 of the treatment device 120 (FIG. 6). Once the guidewire 50 is in position at the treatment site and the therapeutic assembly 100 is in the low-profile delivery configuration, the treatment device 120 can be pulled back with or over the guidewire 50. In further embodiments, a guide catheter or straightening sheath (e.g., shown as delivery element 130 in FIG. 2) can be axially advanced relative to the therapeutic assembly 100 to transform the track element 110 back to the delivery configuration. In one embodiment, for example, the guide catheter or straightening sheath may be advanced until the distal tip of the catheter or sheath is generally aligned with the distal end of the track element 110. In other embodiments, however, the distalmost portion of the catheter or sheath may be advanced to a different location relative to the therapeutic assembly 100 to achieve transformation of the track element 110 back to a low-profile configuration.

Image guidance, e.g., computed tomography (CT), fluoroscopy, intravascular ultrasound (IVUS), optical coherence tomography (OCT), intracardiac echocardiography (ICE), or another suitable guidance modality, or combinations thereof, may be used to aid the clinician's positioning and manipulation of the therapeutic assembly 100. For example, a fluoroscopy system (e.g., including a flat-panel detector, x-ray, or c-arm) can be rotated to accurately visualize and identify the target treatment site. In other embodiments, the treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the catheter 12 and/or the therapeutic assembly 100. Further, in some embodiments, image guidance components (e.g., IVUS, OCT) may be integrated with the catheter 12, the track element 110, the treatment device 120 and/or run in parallel with the catheter 12 to provide image guidance during positioning and removal of the therapeutic assembly 100. For example, image guidance components (e.g., IVUS or OCT) can be coupled to at least one of the therapeutic assembly 100 to provide three-dimensional images of the vasculature proximate the target site to facilitate positioning or deploying the therapeutic assembly 100 within the target renal blood vessel.

FIG. 7 is a block diagram illustrating a method 700 of delivering and deploying the therapeutic assembly 100 described above with reference to FIGS. 1-6 at a target treatment site for modulating renal nerves. In one embodiment, the method 700 can include transluminally delivering a catheter in a low-profile delivery configuration within a renal artery of a human patient (block 702). The catheter can comprise a therapeutic assembly that includes a track element and a treatment device carried by the track element. The treatment device can include a neuromodulation element at a distal portion thereof. The method 700 can also include transforming the catheter from the delivery configuration to a deployed configuration (block 704). In the deployed configuration, the track element has a radially expanded, generally helical shape configured to place the treatment device and at least the neuromodulation element carried thereon proximate to a renal nerve. In one arrangement, the treatment device can include a sleeve with a lumen for receiving the track element therethrough. When the track element is in the radially expanded, helical shape, the sleeve is configured to slide over the stationary track to position the neuromodulation element at one or more treatment locations along the renal artery.

In one embodiment, the catheter can include a guide catheter or straightening sheath configured to hold the track element in the delivery configuration. Accordingly, the step of transforming the catheter can include a step of at least partially withdrawing the guide catheter (or the straightening sheath) to expose the track element within an interior lumen of the renal artery. In certain embodiments, the therapeutic device components as illustrated in FIGS. 1-4B and 6 above can be configured to fit within an 8 Fr guide catheter or smaller (e.g., 7 Fr, 6 Fr, etc.) to access small peripheral vessels. In additional embodiments, and as described above with respect to FIGS. 5 and 6, the method 700 can optionally include delivering the catheter to the renal artery over a guidewire (not shown). In one embodiment, delivering the catheter over a guidewire can also include exchanging the guidewire for the therapeutic assembly within the catheter.

The method 700 can further include modulating the renal nerve (block 706). In this step, energy can be delivered to the renal nerve via the neuromodulation element at a first treatment location along the renal artery. The treatment device can then be moved along the relatively stationary track element to position the neuromodulation element at a second treatment location along the renal artery, and energy can be delivered to the renal nerve via the neuromodulation element at the second treatment location. For example, the treatment device can slide along the relatively stationary track element having the generally helical shape to position the neuromodulation element at the one or more treatment locations along an inner wall of the renal artery. Moving the treatment device transposes the neuromodulation element circumferentially and longitudinally relative to a longitudinal axis of the renal artery. In one embodiment, the neuromodulation element can emit RF energy for modulating the renal nerve adjacent the inner wall of the renal artery at the targeted treatment locations. Delivering energy at the first and second treatment locations can form an interrupted lesion along the inner wall of the renal artery. In other embodiments, however, energy can be delivered during movement of the treatment device to form a continuous lesion along the inner wall. In certain other embodiments, modulation of the renal nerve can occur by delivering energy via the neuromodulation element at a single treatment location.

FIG. 8 is a block diagram illustrating another method 800 of delivering and deploying the therapeutic assembly 100 described above with reference to FIGS. 1-6 at a target treatment site for modulating renal nerves. For example, the method 800 can include intravascularly positioning a therapeutic assembly at a treatment site within a target blood vessel of a human patient (block 802). The therapeutic assembly can comprise a track element in a low-profile delivery configuration and an elongate member over the track element. The elongate member can comprise an electrode disposed a distal portion thereof. The method 800 can also include transforming the track element from the delivery configuration to a deployed configuration, wherein, in the deployed configuration, the track element comprises a spiral track tending to be in apposition with an inner wall of the target blood vessel (block 804). In one embodiment, the track element can be maintained in the delivery configuration with a delivery element (e.g., guide catheter, straightening sheath, etc.), and the step of transitioning the track element can include at least partially withdrawing or retracting the delivery element. The method 800 can further include sliding the elongate member over the track element to position the electrode at a treatment location along the spiral track (block 806). The method 800 can also include delivering energy via the electrode to modulate target nerves proximate to the inner wall of the target blood vessel (block 808).

### Additional Embodiments

Features of the catheter device components described above and illustrated in FIGS. 1-6B can be modified to form additional embodiments configured in accordance with the present technology. For example, neuromodulation system 10 can provide delivery of any of the therapeutic assemblies 100 illustrated in FIGS. 2-4B, 6A, and 6B using one or more additional delivery elements such as guide catheters, straightening sheaths, and/or guidewires. Similarly, the therapeutic assemblies described above and illustrated in FIGS. 1-3B, 6A and 6B showing only a single neuromodulation element can also include additional electrode elements, wires, and energy delivery features positioned along the treatment device.

Various method steps described above for delivery and deployment of the therapeutic assembly components also can be interchanged to form additional embodiments of the present technology. For example, while the method steps described above are presented in a given order, alternative embodiments may perform steps in a different order. The various embodiments described herein may also be combined to provide further embodiments.

### Renal Neuromodulation

Renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and in particular conditions associated with central sympathetic over-stimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, osteoporosis, and sudden death. The reduction of afferent neural signals contributes to the systemic reduction of sympathetic tone/drive, and renal neuromodulation is expected to be useful in treating several conditions associated with systemic sympathetic over activity or hyperactivity. Renal neuromodulation can potentially benefit a variety of organs and bodily structures innervated by sympathetic nerves.

Various techniques can be used to partially or completely incapacitate neural pathways, such as those innervating the kidney. The purposeful application of energy (e.g., electrical energy, thermal energy) to tissue by energy delivery element(s) or components such as those described in conjunction with the intravascular treatment assemblies above, can induce one or more desired thermal heating effects on localized regions of the renal artery and adjacent regions of the renal plexus, which lay intimately within or adjacent to the adventitia of the renal artery. The purposeful application of the thermal heating effects can achieve neuromodulation along all or a portion of the renal plexus.

The thermal heating effects can include both thermal ablation and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for the ablative thermal alteration.

More specifically, exposure to thermal energy (heat) in excess of a body temperature of about 37° C., but below a temperature of about 45° C., may induce thermal alteration via moderate heating of the target neural fibers or of vascular structures that perfuse the target fibers. In cases where vascular structures are affected, the target neural fibers are denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures. In some patients, it may be desirable to achieve temperatures that thermally ablate the target neural fibers or the vascular structures, but that are less than about 90° C., or less than about 85° C., or less than about 80° C., and/or less than about 75° C. Regardless of the type of heat exposure utilized to induce the thermal neuromodulation, a reduction in renal sympathetic nerve activity (RSNA) is expected.

### Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method, comprising:
intravascularly positioning a therapeutic assembly at a treatment site within a target blood vessel of a human patient, wherein the therapeutic assembly comprises a track element in a low-profile delivery configuration and an elongate member about the track element, and wherein the elongate member comprises an electrode disposed at a distal portion thereof;
transforming the track element from the delivery configuration to a deployed configuration such that the track element comprises a spiral track tending to be in apposition with an inner wall of the target blood vessel;
sliding the elongate member along the track element to position the electrode at a treatment location along the spiral track; and
delivering energy via the electrode to modulate target nerves proximate to the inner wall of the target blood vessel.

2. The method of claim 1 wherein the treatment location comprises a first treatment location, and wherein, after delivering energy at the first treatment location, the method further comprises:
sliding the elongate member along the track element such that the electrode is positioned at a second treatment location along the spiral track, the second treatment location being circumferentially and longitudinally displaced relative to the first treatment location; and
delivering energy via the electrode to modulate target nerves proximate to the inner wall of the target blood vessel at the second treatment location.

3. The method of claim 1, further comprising:
transforming the track element from the deployed configuration to the delivery configuration after delivering energy; and
removing the therapeutic assembly from the patient.

4. The method of claim 1 wherein the track element is a nitinol wire, and wherein the elongate member is configured to slide over and along the nitinol wire to position the electrode at a plurality of treatment locations along the spiral track.

5. The method of claim 1 wherein:
intravascularly positioning the therapeutic assembly includes delivering the therapeutic assembly through a guide catheter, wherein the guide catheter is configured to restrain the track element in the delivery configuration; and
transforming the track element from the delivery configuration to a deployed configuration comprises withdrawing the guide catheter in a proximal direction until the track element recovers from the low-profile delivery configuration to the deployed configuration within the target blood vessel.

6. The method of claim 1 wherein the track element is maintained in the delivery configuration with a straightening sheath, and wherein transforming the track element from the delivery configuration to a deployed configuration comprises at least partially retracting the straightening sheath relative to the track element.

7. The method of claim 1 wherein:
intravascularly positioning a therapeutic assembly at a treatment site within a target blood vessel comprises positioning the therapeutic assembly within a renal artery of the patient; and
delivering energy via the electrode comprises delivering thermal radio frequency (RF) energy via the electrode to modulate renal nerves adjacent the renal artery.

8. A method for renal neuromodulation, the method comprising:
transluminally delivering a catheter in a low-profile delivery configuration within a renal artery and proximate to a renal nerve of a human patient, wherein the catheter comprises a track element and a treatment device carried by the track element, and wherein the treatment device includes a neuromodulation element at a distal portion thereof;
transforming the catheter from the delivery configuration to a deployed configuration, wherein the track element has a radially expanded, helical shape configured to place the treatment device distal portion and the neuromodulation element proximate to the renal nerve;
modulating the renal nerve by
delivering energy to the renal nerve via the neuromodulation element at a first treatment location along the renal artery;
moving the treatment device along the track element to position the neuromodulation element at a second treatment location along the renal artery; and
delivering energy to the renal nerve via the neuromodulation element at the second treatment location.

9. The method of claim 8, further comprising delivering a guidewire to the renal artery prior to delivering a catheter, and wherein delivering a catheter comprises passing the treatment device over the guidewire to the renal artery, then exchanging the guidewire for the track element within the treatment device.

10. The method of claim 8 wherein:
the treatment device includes a sleeve with a lumen therethrough;
the neuromodulation element is located at a distal portion of the sleeve; and
the track element is received in the lumen,
wherein, when the catheter is in the deployed configuration, the sleeve is configured to slidably move over the radially-expanded helically-shaped track to position the neuromodulation element at the first and second treatment locations along the renal artery.

11. The method of claim 8, further comprising:
moving the treatment device along the track element to position the neuromodulation element at one or more additional treatment locations along the renal artery; and
delivering neuromodulatory energy via the neuromodulation element at each additional treatment location.

12. The method of claim 8 wherein moving the treatment device transposes the neuromodulation element circumferentially and longitudinally relative to a longitudinal axis of the renal artery.

13. The method of claim 8 wherein delivering energy at the first and second treatment locations forms an interrupted lesion along an inner wall of the renal artery.

14. The method of claim 8 wherein the track element comprises a radially expandable wire.

15. An apparatus for neuromodulation, the apparatus comprising:
a catheter configured for intravascular placement within a target blood vessel of a human patient;
a therapeutic assembly at a distal portion of the catheter, wherein the therapeutic assembly comprises a track element and an elongate member slidably carried by the track element, and wherein the elongate member comprises a neuromodulation element disposed about a distal portion thereof;
wherein the track element is configured to self-expand from a low-profile delivery configuration to a deployed configuration having a pre-shaped spiral structure sized and shaped to position the neuromodulation element in apposition with one or more treatment positions along the inner wall of the target blood vessel.

16. The apparatus of claim 15 wherein the track element comprises a nitinol wire, and wherein the elongate member comprises a sheath configured to at least partially cover the nitinol wire.

17. The apparatus of claim 15 wherein the neuromodulation element is a first neuromodulation element, and wherein the elongate member comprises a second neuromodulation element spaced apart from the first neuromodulation element.

18. The apparatus of claim 15 wherein the catheter is a guide catheter, and wherein the guide catheter is configured to radially restrain the track element in the delivery configuration while intravascularly locating the therapeutic assembly in the target blood vessel, and further wherein proximal retraction of the guide catheter relative to the therapeutic assembly releases the track element to the deployed configuration.

19. The apparatus of claim 15 wherein the neuromodulation element comprises an electrode disposed at the distal portion of the elongate member and configured to deliver thermal radio frequency (RF) energy.

20. The apparatus of claim 15, further comprising an operative wire for pushing or pulling the elongate member relative to the track element.
